(19) United States
(12) Patent Application Publication
(10) Pub. No.: US 2010/0304979 A1
(43) Pub. Date: Dec. 2, 2010

(54) Diagnostic Methods and Arrays for Use in the Same

(76) Inventors: Carl Arne Krister Borrebaeck, Lund (SE); Lars Bertil Christer Wingren, Sodra Sandby (SE)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/744,421

(22) PCT Filed: Nov. 24, 2008
(86) PCT No.: PCT/GB08/03922

§ 371 Date: Aug. 23, 2010

(30) Foreign Application Priority Data
Nov. 27, 2007 (GB) 0723179.8

(51) Int. Cl. C40B 30/00; G01N 33/68; G01N 33/566; C40B 40/10

(52) U.S. Cl. 506/7; 436/64; 436/501; 506/18

(57) ABSTRACT

The present invention provides a method for determining the presence of breast cancer cells in an individual comprising the steps of (a) providing a serum or plasma sample to be tested and (b) determining a protein signature of the test sample by measuring the presence and/or amount in the test sample of two or more proteins selected from the group defined in Table 1a or 1b, wherein the presence and/or amount in the test sample of the two or more proteins selected from the group defined in Table 1a or 1b is indicative of the presence of breast cancer cells. In a preferred embodiment, the two or more proteins include IL-5 and/or MCP-3. Also provided are arrays and diagnostic kits for use in the methods of the invention.

### FIELD OF THE INVENTION

The present invention provides methods for use in the diagnosis of breast cancer, as well as arrays and kits for use in such methods.

### BACKGROUND OF THE INVENTION

Early and improved detection and diagnosis of breast cancer, which is worldwide the most common form (about 30%) of cancer in females, is essential^{1, 2}. The recent progress of proteomics has opened up novel avenues for cancer-related biomarker discovery^{3, 4}. However, adopting high-throughput proteomic approaches to multiplexed set-ups, providing a minimally invasive screening procedure, targeting non-fractionated biological fluids, such as blood, has proven to be challenging^{1,2,4}. Antibody-based microarrays represent a rapidly emerging affinity proteomic technology that is likely to play an increasing role within oncoproteomics⁵. In recent years, the technology has made significant progress^{6,7} (for review, see refs 8-11) now allowing us to design miniaturized array platforms, capable of simultaneously profiling numerous low abundant protein analytes in complex proteomes, such as plasma and serum, while consuming only minute amounts of sample^{6,12,13}. Adopting antibody microarrays, translational proteomics is one immediate application where comparative protein expression profiling analysis of cancer vs. normal proteomes could yield tentative predictive biomarker signatures¹²⁻¹⁸. From a clinical point of view, we also need increased possibilities to individually monitor disease progression and response to treatment, since no therapy has the same effect on a large number of patients with the same diagnosis.

To increase the diagnostic and predictive power in cancer, the critical value of using more than one biomarker has already been suggested^{2,19-21}, which now drives the search for specific disease-associated biomarker signatures^{3,4}. This is true, to an even higher degree, for heterogeneous diseases, such as breast cancer, where a single biomarker is unlikely to give conclusive diagnostic information to successfully stratify all the different disease states known. This is illustrated by the fact that altered serum levels of a variety of (single) analytes, such as truncated forms of complement protein C3a²², cancer antigen (CA) 15-3³, carcinoembryonic antigen (CEA)², glycoproteins of the MUC family², autoantibodies², sialyl Lewis^{x 21,23} and cytokines (e.g. IL-6, IL-8, IL-10)²⁴ have been observed in breast cancer patients, using traditional proteomic approaches. Still, these data are inconclusive and the specificity and sensitivity of these tentative single biomarkers are too low.

Recently, the first monoclonal antibody-based microarrays have been applied to analyze breast cancer cell lines, resulting in the identification of IL-8 as tentative key factor suggested in breast cancer invasion and progression²⁵⁻²⁷, IL-8/GRO as a HER2 (erB-2-)-induced cytokine signature²⁸, as well as a five protein signature that may be associated with doxorubicin resistance²⁹. Similarly, Hudelist and co-workers have used monoclonal antibody microarrays to detect a set of differentially expressed proteins in normal vs. malignant breast tissue from one patient³⁰.

However, there remains a need for improved methods for the diagnosis of breast cancer.

### DISCLOSURE OF THE INVENTION

A first aspect of the invention provides a method for determining the presence of breast cancer cells in an individual comprising the steps of:
(a) providing a serum or plasma sample to be tested; and
(b) determining a protein signature of the test sample by measuring the presence and/or amount in the test sample of two or more proteins selected from the group defined in Table 1a or 1b,
   wherein the presence and/or amount in the test sample of one or more proteins selected from the group defined in Table 1a or 1b is indicative of the presence of breast cancer cells.

TABLE 1a
Biomarkers for the diagnosis of breast cancer

| Abbreviation | Full name | Exemplary sequences* |
|---|---|---|
| C3 | Complement | BC150179, BC150200, P01024 |
| | protein 3 | |
| C4 | Complement | BC151204, BC146673, AB209989, |
| | protein 4 | AY379959, AL645922, AY379927, |
| | | AY379926, AY379925, P0C0L4, |
| | | P0C0L5 |
| C5 | Complement | BC113738, BC113740, DQ400449, |
| | protein 5 | AB209031, P01031 |
| IL-8 | Interleukin-8 | CR623827, CR623683, DQ893727, |
| | | DQ890564, P10145 |
| Sialyl Le^{x} | Sialyl Lewis^{x} | N/A |
| IL-5 | Interleukin-5 | BC066282, CH471062, P05113 |
| IL-7 | Interleukin-6 | AK226000, AB102893, AB102885, |
| | | P13232 |
| MCP-3 | Monocyte | BC112258, BC112260, BC092436, |
| | chemotactic | BC070240, P80098 |
| | protein 3 | |
| TM peptide** | 10TM protein | N/A |

*It will be appreciated by persons skilled in the art that the present invention is not limited to the detection of proteins having these exemplary amino acid sequences, but encompasses the detection of naturally-occurring (e.g. allelic) variants of such sequences.
**This peptide is the antigen to which the scFv antibody construct of SEQ ID NO: 1 binds (see below).

TABLE 1b
Further biomarkers for the diagnosis of breast cancer

| | | Exemplary |
|---|---|---|
| Abbreviation | Full name | sequences* |
| IL-3 | Interleukin 3 | P08700 |
| TNFβ | Tumour necrosis factor beta | P01374 |

In one embodiment, the two or more proteins measured in step (b) of the method of the invention include IL5 and/or MCP-3.

By “breast cancer” we include both sporadic and hereditary breast cancer. In one embodiment, the methods provides for the diagnosis of metastatic breast cancer.

By “protein signature” we include the meaning of a combination of the presence and/or amount of a plurality of proteins present in a serum/plasma sample from an individual having breast cancer, which protein combination can be distinguished from a combination of the presence and/or amount of proteins present in a serum/plasma sample from a normal, or healthy, individual (i.e. not suffering from cancer).

The serum or plasma sample provided in step (a) is typically derived from a blood sample, and may be prepared using methods well known in the art. The sample may be in a native state or a digested format, depending on the method used to detect the proteins therein.

As detailed in the accompanying Examples, the presence and/or amount of certain serum proteins present in a test sample may be indicative of the presence of cancer, such as breast cancer, in an individual. For example, the relative presence and/or amount of certain serum proteins in a single test sample may be indicative of the presence of cancer, such as breast cancer, in an individual.

The individual being tested is typically a human. However, it will be appreciated that the methods may also be used for the diagnosis of any domestic or farm mammal (such as a horse, pig, cow, sheep, dog or cat).

In one embodiment, the method of the first aspect of the invention further comprises the steps of:
(c) providing a control serum or plasma sample from a healthy individual (not suffering from breast cancer); and
(d) determining a protein signature of the control sample by measuring the presence and/or amount in the control sample of the two or more proteins measured in step (b);
   wherein the presence of breast cancer cells is identified in the event that the presence and/or amount in the test sample of the two or more proteins measured in step (b) is different from the presence and/or amount in the control sample of the one or more proteins measured in step (b).

Such steps may be performed before, during or after steps (a) and (b).

Preferably, the healthy individual is age- and/or sex-matched for the individual to be tested. In other words, the healthy individual is approximately the same age (e.g. within 5 years) and is the same sex as the individual to be tested.

In an alternative embodiment, the presence and/or amount in the test sample of the one or more proteins measured in step (b) are compared against predetermined reference values (which correspond to healthy individuals).

In either embodiment, it is preferred if the presence and/or amount in the test sample of the one or more proteins measured in step (b) is significantly different (i.e. statistically different) from the presence and/or amount of the one or more proteins measured in step (d) or the predetermined reference values. For example, as discussed in the accompanying Examples, significant difference between the presence and/or amount of a particular protein in the test and control samples may be classified as those where p<0.05.

Typically, step (b) comprises measuring the presence and/or amount in the test sample of at least three proteins selected from the group defined in Table 1a or 1b, for example at least four, five, six, seven, eight, nine or ten proteins selected from the group defined in Table 1a or 1b.

For example, step (b) may comprise measuring the presence and/or amount in the test sample of at least one protein selected from the group consisting of IL-5, IL-7, MCP-3 and TM peptide. Thus, step (b) may comprise measuring the presence and/or amount in the test sample of at least two proteins selected from the group consisting of IL-5, IL-7, MCP-3 and TM peptide, for example three or four proteins.

By “TM peptide” we mean a peptide derived from a 10TM protein, to which the scFv antibody construct of SEQ ID NO:1 below has specificity (wherein the CDR sequences are underlined):

[SEQ ID NO: 1]
MAEVQLLESGGGLVQPGGSLRLSCAASGFTFSSYGFHWVRQAPGKGLEWV
SLISWDGGSTYYADSVKGRFTISRDNSKNTLYLQMNSLRAEDTAVYYCAR
GTWFDPWGQGTLVTVSSGGGGSGGGGSGGGGSQSVLTQPPSASGTPGQRV
TISCSGSSSNIGNNAVNWYQQLPGTAPKLLIYRNNQRPSGVPDRFSGSKS
GGTSASLAISGLRSEDEADYYCAAWDDSLSWVFGGGTKLTVLG

Hence, this scFv may be used or any antibody, or antigen binding fragment thereof, that competes with this scFv for binding to the 10TM protein. For example, the antibody, or antigen binding fragment thereof, may comprise the same CDRs as present in SEQ ID NO:1.

It will be appreciated by persons skilled in the art that such an antibody may be produced with an affinity tag (e.g. at the C-terminus) for purification purposes. For example, an affinity tag of SEQ ID NO:2 below may be utilised:

| | |
|---|---|
| DYKDHDGDYKDHDIDYKDDDDKAAAHHHHHH | [SEQ ID NO: 2] |

In a further embodiment, step (b) may comprise measuring the presence and/or amount in the test sample of IL-5 and MCP-3.

Alternatively, or in addition, step (b) may comprise measuring the presence and/or amount in the test sample of at least one protein selected from the group consisting of C3, C4, C5, IL-8 and sialyl Lewis^{x}. Thus, step (b) may comprise measuring the presence and/or amount in the test sample of at least two proteins selected from the group consisting of C3, C4, C5, IL-8 and sialyl Lewis^{x}, for example three, four or five proteins.

Step (b) may further comprise measuring the presence and/or amount in the test sample of at least one of the proteins defined in Table 1b. Thus, in one embodiment, step (b) comprises measuring the presence and/or amount in the test sample of all of the proteins defined in Table 1b.

In a most preferred embodiment of the first aspect of the invention, step (b) comprises measuring the presence and/or amount in the test sample of all nine of the proteins, defined in Table 1a or 1b.

A positive diagnosis of breast cancer may be indicated by the presence or an increase in the amount (relative to the healthy control or predetermined reference values) of one or more of C3, C4, C5, IL-8 and sialyl Lewis^{x}. Alternatively, or in addition, a positive diagnosis of breast cancer may be indicated by the absence or a decrease in the amount (relative to the healthy control or predetermined reference values) of one or more of IL-5, IL-7, MCP-3, TM peptide.

Advantageously, the presence or an increase in the amount of one or more of C3, C4, C5, IL-8, sialyl Lewis^{x} and IL-3 and/or the absence or a decrease in the amount of one or more of IL-5, IL-7, MCP-3, TM peptide and TNF-β (relative to the healthy control or predetermined reference values) may be indicative of a positive diagnosis of breast cancer in the individual being tested.

In one embodiment, the method of the first aspect of the invention further comprises the step of determining the medication history of the individual. This may involve or consist of determining whether the patient has taken any anti-inflammatory drugs and/or hormones prior to providing the serum or plasma sample to be tested.

Thus, the methods of the invention may be used to diagnose a patient who has not taken any anti-inflammatory drugs and/or hormones prior to providing the serum or plasma sample (for example, within one week or one, two, three, four, five, six or more months of the date on which the sample is collected).

When such a patient is tested, step (b) preferably comprises determining a protein signature of the test sample by measuring the presence and/or amount in the test sample of one or more proteins selected from the group consisting of TNF-β, IL-12, C4, MCP-1, IL-3, IL-7, integrin α10 and IL-4. For example, step (b) may comprise determining the protein signature of the test sample by measuring the presence and/or amount in the test sample of two or more proteins selected from the group consisting of TNF-β, IL-12, C4, MCP-1, IL-3, IL-7, integrin α10 and IL-4, for example at least three, four, five, six or seven proteins. Most preferably, step (b) comprises determining a protein signature of the test sample by measuring the presence and/or amount in the test sample of all of the proteins from the group consisting of TNF-β, IL-12, C4, MCP-1, IL-3, IL-7, integrin α10 and IL-4.

In one embodiment of the methods of the invention described above, step (b) and/or step (d) may be performed using label-free detection technologies, such as surface plasmon resonance, surface plasmon resonance imaging or mass spectrometry (single or tandem). The application of such methods to the detection of proteins and peptides is well known in the art.

In a further embodiment of the methods of the invention described above, step (b) and/or step (d) may be performed using first binding agents capable of binding to the two or more proteins (which may be immobilised on the surface of a test substrate, such as a microarray).

Suitable binding agents (also referred to as binding molecules) may be selected or screened from a library based on their ability to bind a given protein or motif, as discussed below.

Preferably, one or more of the first binding agents is an antibody (such as an IgG molecule) or an antigen-binding fragment thereof. Conveniently, the antibody or fragment thereof is a monoclonal antibody or an antigen-binding fragment thereof.

The term “antibody” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecules capable of binding to an antigen in an immunoassay format that is known to those skilled in the art.

We also include the use of antibody-like binding agents, such as affibodies and aptamers.

A general review of the techniques involved in the synthesis of antibody fragments which retain their specific binding sites is to be found in Winter & Milstein (1991) Nature 349, 293-299.

Additionally, or alternatively, one or more of the first binding molecules may be an aptamer (see Collett et al., 2005, Methods 37:4-15).

Molecular libraries such as antibody libraries (Clackson et al., 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

The molecular libraries may be expressed in vivo in prokaryotic cells (Clackson et al, 1991, op. cit.; Marks et al, 1991, op. cit.) or eukaryotic cells (Kieke et al, 1999, Proc Natl Acad Sci USA, 96(10):5651-6) or may be expressed in vitro without involvement of cells (Hanes & Pluckthun, 1997, Proc Natl Acad Sci USA 94(10):4937-42; He & Taussig, 1997, Nucleic Acids Res 25(24):5132-4; Nemoto et al, 1997, FEBS Lett, 414(2):405-8).

In cases when protein based libraries are used, the genes encoding the libraries of potential binding molecules are often packaged in viruses and the potential binding molecule displayed at the surface of the virus (Clackson et al, 1991, supra; Marks et al, 1991, supra; Smith, 1985, supra).

Perhaps the most commonly used display system is filamentous bacteriophage displaying antibody fragments at their surfaces, the antibody fragments being expressed as a fusion to the minor coat protein of the bacteriophage (Clackson et al, 1991, supra; Marks et al, 1991, supra). However, other suitable systems for display include using other viruses (EP 39578), bacteria (Gunneriusson et al, 1999, supra; Daugherty et al, 1998, Protein Eng 11(9):825-32; Daugherty et al, 1999, Protein Eng 12(7):613-21), and yeast (Shusta et al, 1999, J Mol Biol 292(5):949-56).

In addition, display systems have been developed utilising linkage of the polypeptide product to its encoding mRNA in so-called ribosome display systems (Hanes & Pluckthun, 1997, supra; He & Taussig, 1997, supra; Nemoto et al, 1997, supra), or alternatively linkage of the polypeptide product to the encoding DNA (see U.S. Pat. No. 5,856,090 and WO 98/37186).

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

Thus, the antibody or antigen-binding fragment may be selected from the group consisting of intact antibodies, Fv fragments (e.g. single chain Fv [scFv] and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]).

By “scFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

When potential binding molecules are selected from libraries, one or more selector peptides having defined motifs are usually employed. Amino acid residues that provide structure, decreasing flexibility in the peptide or charged, polar or hydrophobic side chains allowing interaction with the binding molecule may be used in the design of motifs for selector peptides. For example:
(i) Proline may stabilise a peptide structure as its side chain is bound both to the alpha carbon as well as the nitrogen;
(ii) Phenylalanine, tyrosine and tryptophan have aromatic side chains and are highly hydrophobic, whereas leucine and isoleucine have aliphatic side chains and are also hydrophobic;
(iii) Lysine, arginine and histidine have basic side chains and will be positively charged at neutral pH, whereas aspartate and glutamate have acidic side chains and will be negatively charged at neutral pH;
(iv) Asparagine and glutamine are neutral at neutral pH but contain a amide group which may participate in hydrogen bonds;
(v) Serine, threonine and tyrosine side chains contain hydroxyl groups, which may participate in hydrogen bonds.

Typically, selection of binding molecules may involve the use of array technologies and systems to analyse binding to spots corresponding to types of binding molecules.

It will be appreciated by persons skilled in the art that the one or more proteins in the test sample may be labelled with a detectable moiety prior to performing step (b) and/or step (d).

By a “detectable moiety” we include a moiety which permits its presence and/or relative amount and/or location (for example, the location on an array) to be determined, either directly or indirectly.

Suitable detectable moieties are well known in the art.

For example, the detectable moiety may be a fluorescent and/or luminescent and/or chemiluminescent moiety which, when exposed to specific conditions, may be detected. Such a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

In a further embodiment, the detectable moiety may be a radioactive atom which is useful in imaging. Suitable radioactive atoms include ^{99m}Tc and ¹²³I for scintigraphic studies. Other readily detectable moieties include, for example, spin labels for magnetic resonance imaging (MRI) such as ¹²³I again, ¹³¹I, ¹¹¹In, ¹⁹F, ¹³C, ¹⁵N, ¹⁷O, gadolinium, manganese or iron. Clearly, the agent to be detected (such as, for example, the one or more proteins in the test sample and/or control sample described herein and/or an antibody molecule for use in detecting a selected protein) must have sufficient of the appropriate atomic isotopes in order for the detectable moiety to be readily detectable.

The radio- or other labels may be incorporated into the proteins present in the samples of the methods of the invention and/or the binding agents of the invention in known ways. For example, if the binding agent is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate ¹²³I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

It will be appreciated by persons skilled in the art that proteins in the sample(s) to be tested may be labelled with a moiety which indirectly assists with determining the presence, amount and/or location of said proteins. Thus, the moiety may constitute one component of a multicomponent detectable moiety. For example, the proteins in the sample(s) to be tested may be labelled with biotin, which allows their subsequent detection using streptavidin fused or otherwise joined to a detectable label.

In a further embodiment of the first aspect of the invention, step (b) and/or step (d) of the method are performed using an array.

Arrays per se are well known in the art. Typically they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. Alternatively, affinity coupling of the probes via affinity-tags or similar constructs may be employed. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7(18 Suppl):S143-9).

Typically the array is a microarray. By “microarray” we include the meaning of an array of regions having a density of discrete regions of at least about 100/cm², and preferably at least about 1000/cm². The regions in a microarray have typical dimensions, e.g. diameter, in the range of between about 10-250 μM, and are separated from other regions in the array by about the same distance. The array may alternatively be a macroarray or a nanoarray.

Once suitable binding molecules (discussed above) have been identified and isolated, the skilled person can manufacture an array using methods well known in the art of molecular biology; see Examples below.

In a further embodiment of the methods of the invention, step (b) and/or step (d) may be performed using an assay comprising a second binding agent capable of binding to the one or more proteins, the second binding agent also comprising a detectable moiety. Suitable second binding agents are described in detail above in relation to the first binding agents.

Thus, the proteins of interest in the sample to be tested may first be isolated and/or immobilised using the first binding agent, after which the presence and/or relative amount of said proteins may be determined using a second binding agent.

In one embodiment, the second binding agent is an antibody or antigen-binding fragment thereof; typically a recombinant antibody or fragment thereof. Conveniently, the antibody or fragment thereof is selected from the group consisting of: scFv; Fab; a binding domain of an immunoglobulin molecule. Suitable antibodies and fragments, and methods for making the same, are described in detail above.

Alternatively, the second binding agent may be an antibody-like binding agent, such as an affibody or aptamer.

Alternatively, where the detectable moiety on the protein in the sample to be tested comprises or consists of a member of a specific binding pair (e.g. biotin), the second binding agent may comprise or consist of the complimentary member of the specific binding pair (e.g. streptavidin).

Where a detection assay is used, it is preferred that the detectable moiety is selected from the group consisting of: a fluorescent moiety; a luminescent moiety; a chemiluminescent moiety; a radioactive moiety; an enzymatic moiety. Examples of suitable detectable moieties for use in the methods of the invention are described above.

Preferred assays for detecting serum or plasma proteins include enzyme linked immunosorbent assays (ELISA), radioimmunoassay (RIA), immunoradiometric assays (IRMA) and immunoenzymatic assays (IEMA), including sandwich assays using Monoclonal and/or polyclonal antibodies. Exemplary sandwich assays are described by David et al in U.S. Pat. Nos. 4,376,110 and 4,486,530, hereby incorporated by reference. Antibody staining of cells on slides may be used in methods well known in cytology laboratory diagnostic tests, as well known to those skilled in the art.

Thus, in one embodiment the assay is an ELISA (Enzyme Linked Immunosorbent Assay) which typically involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemiluminescent systems based on enzymes such as luciferase can also be used.

Conjugation with the vitamin biotin is frequently used since this can readily be detected by its reaction with enzyme-linked avidin or streptavidin to which it binds with great specificity and affinity.

In an alternative embodiment, the assay used for protein detection is conveniently a fluorometric assay. Thus, the detectable moiety of the second binding agent may be a fluorescent moiety, such as an Alexa fluorophore (for example Alexa-647).

A second aspect of the invention provides an array for use in a method according to the first aspect of the invention, the array comprising two or more first binding agents as defined above.

In one embodiment, the two or more first binding agents bind to IL5 and/or MCP-3.

Preferably, the array comprises or consists of a plurality of first binding agents which (collectively) are capable of binding to all of the proteins defined in Table 1a or 1b, which binding agents are immobilised.

Arrays suitable for use in the methods of the invention are discussed in detail above.

A third aspect of the invention provides the use of two or more proteins selected from the group defined in Table 1a or ab in combination as a diagnostic co-markers for determining the presence of breast cancer cells in an individual.

In one embodiment, the two or more proteins include IL5 and/or MCP-3.

Advantageously, all of the proteins defined in Table 1a or 1b are used collectively as diagnostic co-markers (i.e. as a protein signature) for determining the presence of breast cancer cells in an individual.

A fourth aspect of the invention provides a diagnostic kit for use in a method according to the first aspect of the invention, the kit comprising or consisting of:
(a) an array according to the second aspect of the invention; and
(b) instructions for performing the method according to the first aspect of the invention (optional).

In one embodiment, the kit further comprises one or more second binding agents as defined above.

It will be appreciated that the kits of the invention may further comprise one or more controls samples, such as a ‘negative control’ sample of proteins obtained or derived from healthy individual and/or ‘positive control’ samples of proteins obtained or derived from an individual with breast cancer.

Preferred, non-limiting examples which embody certain aspects of the invention will now be described, with reference to the following figures:

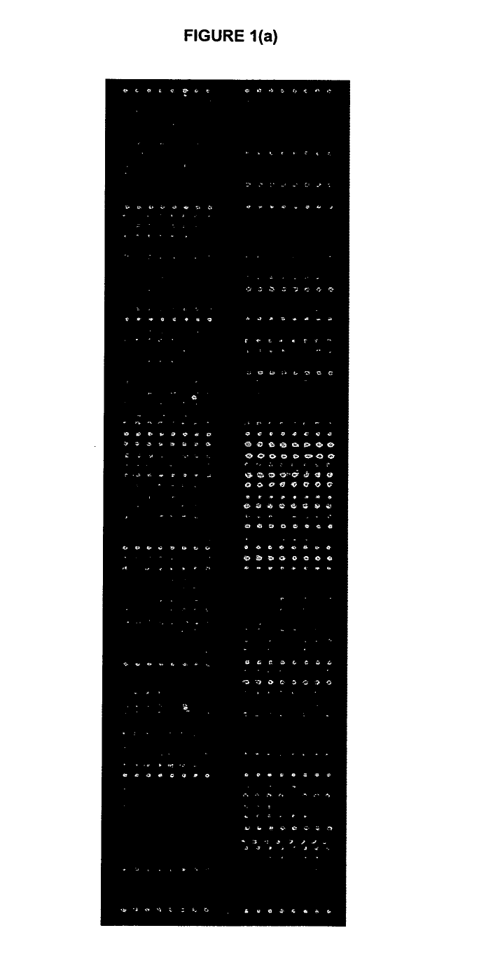

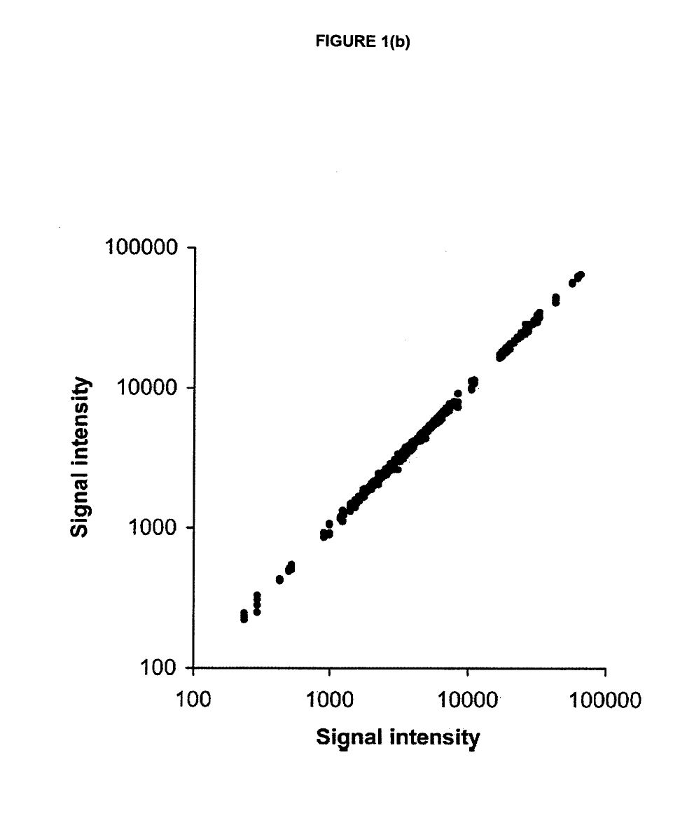

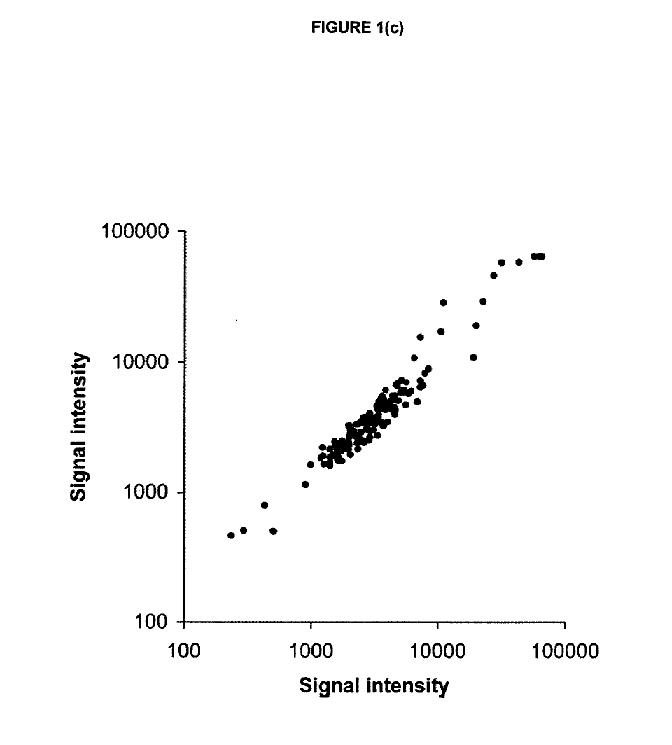

FIG. 1. Evaluation of recombinant scFv antibody microarrays. (A) A scanned representative microarray image of a metastatic breast cancer sample containing 1280 data points. (B) Intraassay reproducibility, i.e. spot-to-spot-variations. The correlation coefficient was found to be 0.99. (C) Inter-assay reproducibility, i.e. reproducibility of duplicate experiments. The correlation coefficient was found to be 0.96.

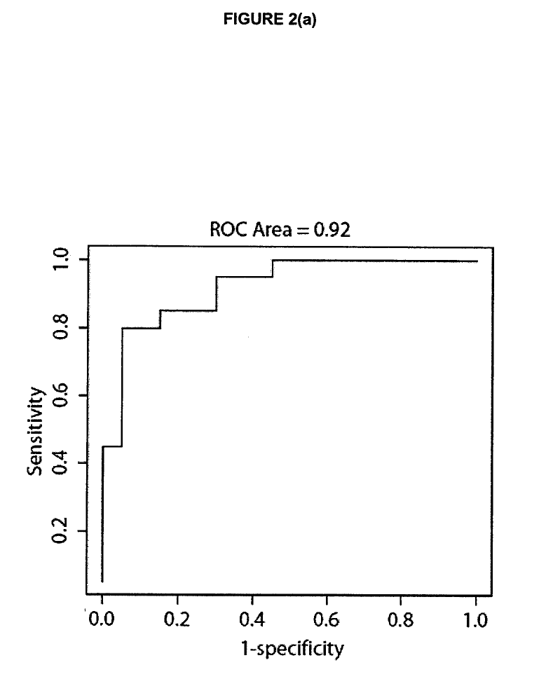

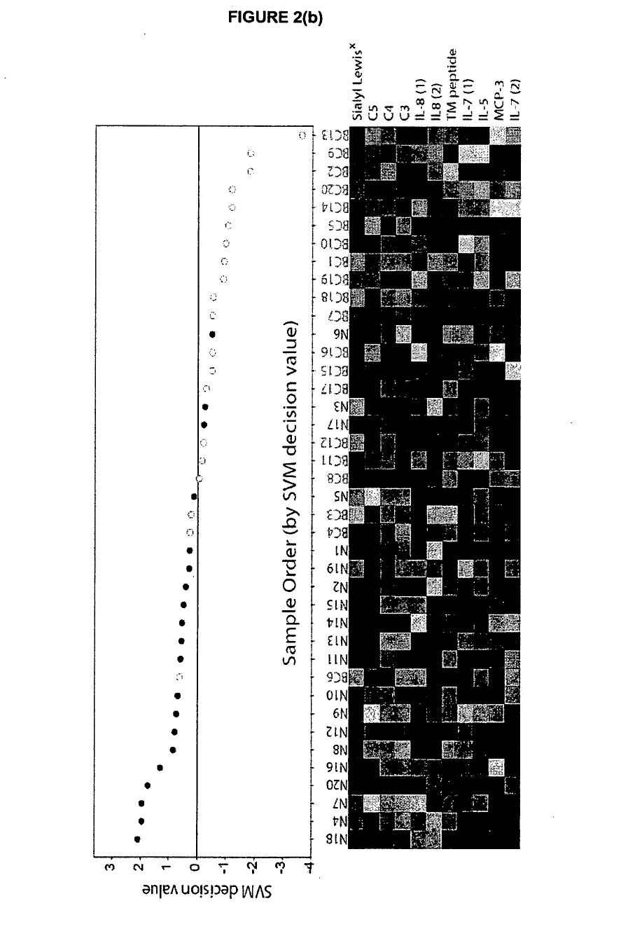

FIG. 2. Classification of metastatic breast cancer patients by serum protein profiling, using recombinant scFv antibody microarrays. (A) A Receiver Operator Characteristics (ROC) curve obtained for metastatic breast cancer patients (n=20) vs. healthy controls (n=20) based on all 129 analytes, using a leave-one-out cross validation approach with a Support Vector Machine (SVM). (B) Classification of the serum samples, using the SVM prediction values based on all 129 analytes. A heat map where the 11 highest ranked, i.e. significantly differentially expressed, analytes, corresponding to 9 non-redundant serum analytes, are hierarchically clustered is shown. (C) The signal intensities observed for the top 3 differentially expressed analytes, C4, IL-8 and C5. The mean values are indicated.

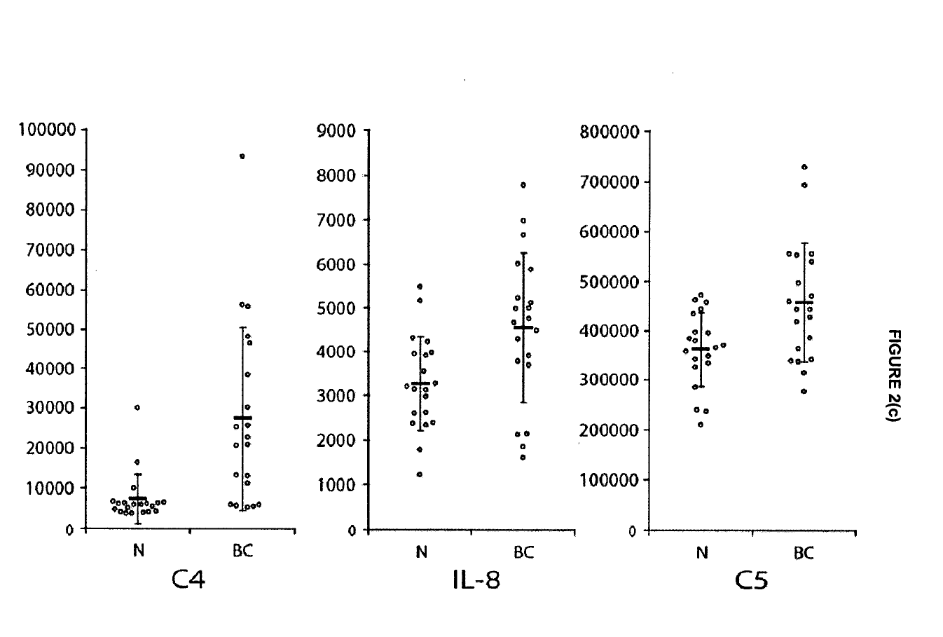

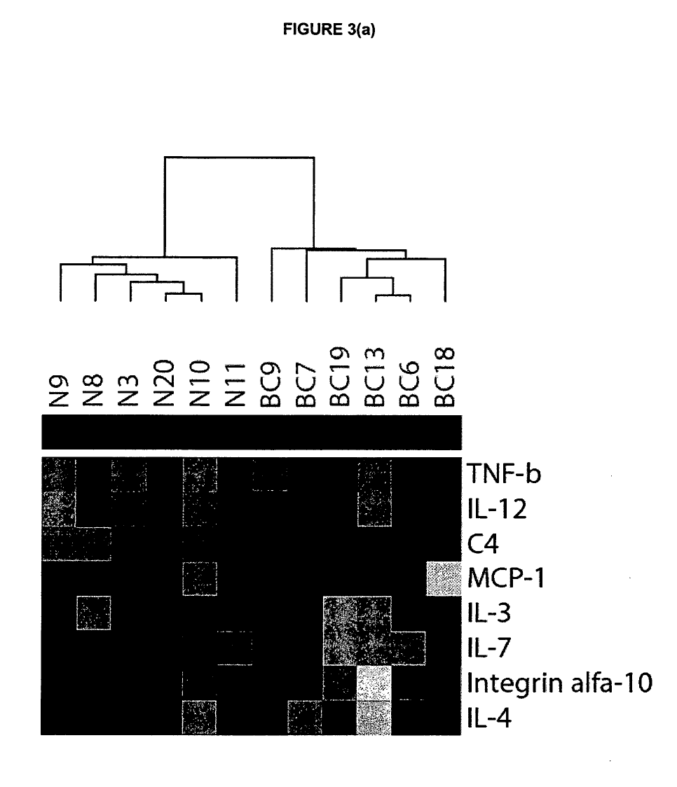

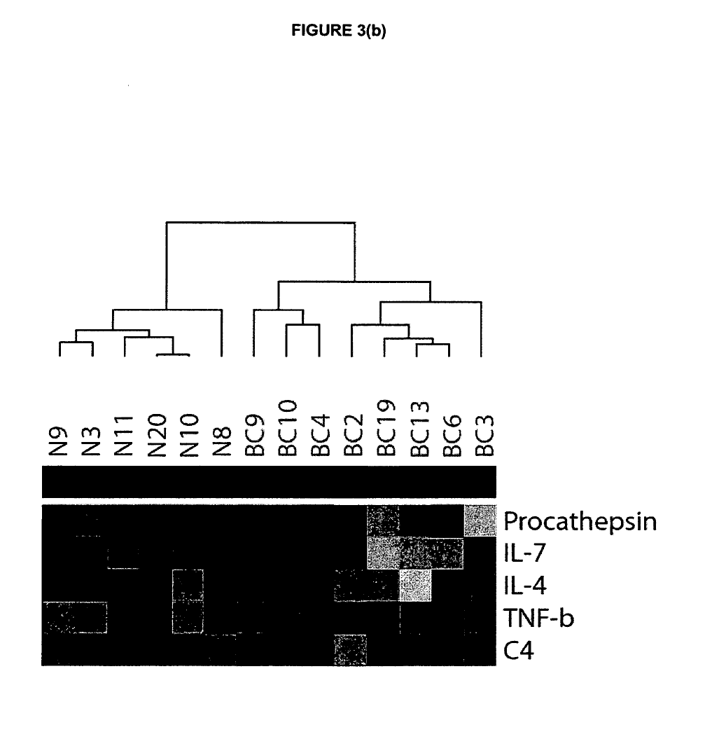

FIG. 3. Effect of drugs and clinical parameters on the classification of metastatic breast cancer patients. (A) Classification of metastatic breast cancer patients (n=6) and healthy controls (n=6) not taking any drugs (anti-inflammatory drugs and/or hormones) at the time when the serum samples were collected. Differentially expressed analytes (p<0.05) were identified using Wilcoxon test, and visualized in a heat map by unsupervised hierarchical clustering. (B) Classification of metastatic breast cancer patients, stages III and IV (n=8) and healthy controls (n=6). Differentially expressed analytes (p<0.05) were identified, using Wilcoxon test, and visualized in a heat map by unsupervised hierarchical clustering.

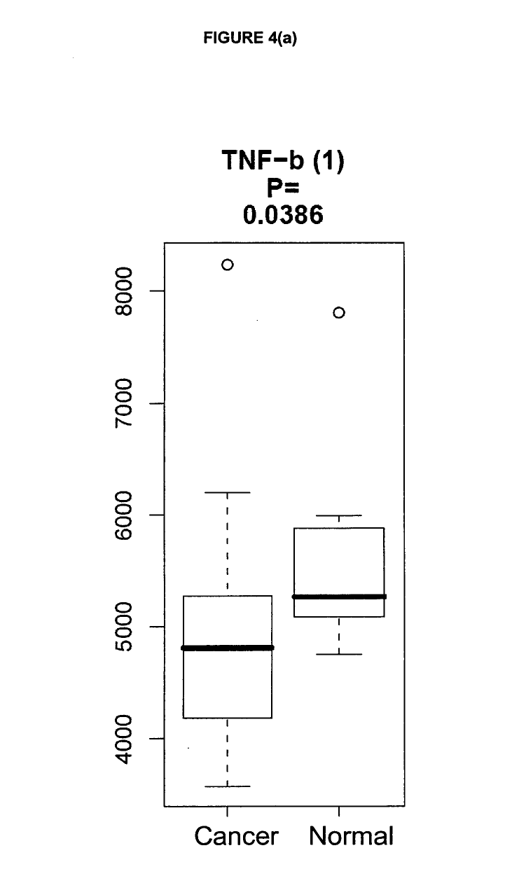

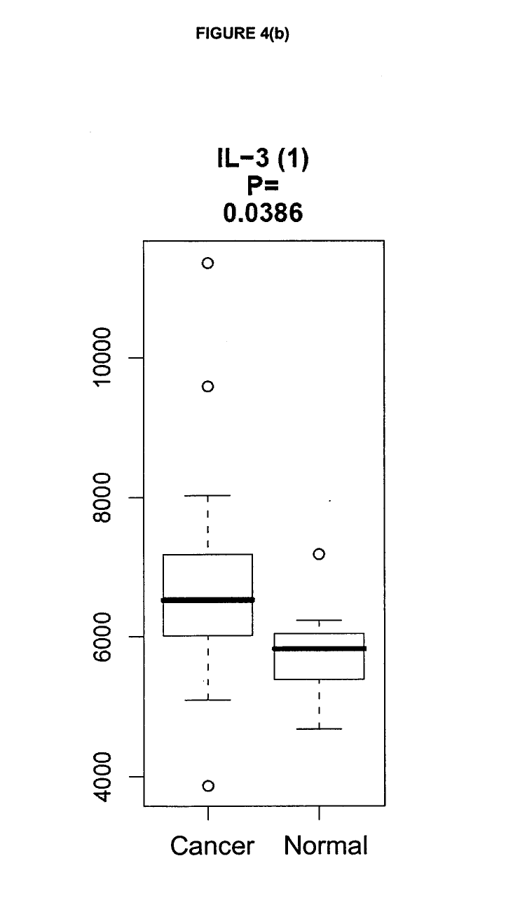

FIG. 4. Differential expression levels (P<0.05) of (a) TNF-β and (b) IL-3 when comparing cohorts of metastatic breast cancer patients vs non-metastatic breast cancer patients.

### EXAMPLES

In the present study, we have for the first time identified the potential of large-scale recombinant scFv antibody microarrays to classify female, post-menopausal, age-matched metastatic breast cancer patients (n=20) vs. healthy controls (n=20) based on differential serum protein profiling. This miniaturized set-up provided unique means to profile even pM range analytes, including mainly immunoregulatory proteins, in non-fractionated sera, while consuming only μL amounts of the clinical samples^{6,7,31}. The results demonstrate that several differentially expressed serum proteins could be detected, and that this breast cancer-associated biomarker signature could be used to classify metastatic breast cancer patients.

### Materials and Methods

### Samples

In total, 40 serum samples, supplied by the Department of Oncology, Lund University Hospital, Lund, Sweden were included in this study. The serum samples were collected from female, post-menopausal patients, 20 of which suffered from metastatic breast cancer (denoted BC1 to BC20) and 20 healthy matched control subjects (denoted N1 to N20). Patient demographics and information about additional clinical parameters, including estrogen receptor (ER) status, progesterone receptor (PgR) status, clinical stage, histological type and histological grade, as well as information about intake of anti-inflammatory drugs/hormones, are shown in Table 2.

TABLE 2
Patient demographics and clinical parameters

Class

| | | BC | N |
|---|---|---|---|
| No. of samples | | 20 | 20 |
| Age | Mean | 60 | 57 |
| | Range | 48-75 | 47-60 |
| ER status | ER+ | 15 | — |
| | ER− | 3 | — |
| | n.d. | 2 | — |
| PgR status | PgR+ | 7 | — |
| | PgR− | 10 | — |
| | n.d. | 3 | |
| Clinical | I | 2 | — |
| stage^{a)} | II | 8 | — |
| | III | 5 | — |
| | IV | 3 | — |
| | n.d. | 2 | — |
| Histological | I | 1 | — |
| grade | II | 8 | — |
| | II-III | 3 | — |
| | III | 8 | — |
| Histological | ductal | 12 | — |
| type | lobular | 4 | — |
| | mixed | 2 | — |
| | n.d. | 2 | — |
| Drugs^{b)} | yes | 14 | 14 |
| | no | 6 | 6 |

^{a)}Refers to the stage at primary diagnosis.
^{b)}All BC patients were treated with a range of various agents (data not shown). Here, only those BC patients (and healthy controls) that had taken any anti-inflammatory drugs and/or hormones at the time when the serum samples were collected are defined.
ER = Estrogen receptor
PgR = Progesterone receptor
n.d. = not determined

### Labelling

The serum samples were labelled, using previously optimized labelling protocols for serum proteomes^{6,7,31}. All serum samples were biotinylated, using EZ-Link Sulfo-NHS-LC-Biotin (Pierce, Rockford, Ill., USA). 50 μl serum aliquots were centrifuged at 16,000×g for 20 minutes at 4° C. and diluted 1:45 in PBS, resulting in a final protein concentration of about 2 mg/ml. Sulfo-NHS-biotin was then added to a final concentration of 10 mM and the samples were incubated on ice for 2 h, with careful vortexing every 20 min. Unreacted biotin was removed by dialysis against PBS for 72 h at 4° C. Finally, the samples were aliquoted and stored at −20° C. prior to use.

Production and Purification of scFv

129 human recombinant scFv antibody fragments against 60 different proteins mainly involved in immune regulation (Table 3), were stringently selected from the n-CoDeR library³², and kindly provided by BioInvent International AB, Lund, Sweden. Hence, some antigens were recognized by up to 4 different scFv clones. All scFv probes were produced in 100 ml E. coli cultures and purified, from either expression supernatants or periplasmic preparations, using affinity chromatography on Ni-NTA agarose (Qiagen, Hilden, Germany). Bound molecules were eluted with 250 mM immidazole, extensively dialyzed against PBS, and stored at 4° C. until further use. The protein concentration was determined by measuring the absorbance at 280 nm (average concentration 210 μg/ml, range 60-1090 μg/ml). The degree of purity and integrity of the scFv antibodies were evaluated by 10% SDS-PAGE (Invitrogen, Carlsbad, Calif., USA).

TABLE 3
The different scFv specificities used for the antibody microarrays

| Antigen | Antigen |
|---|---|
| (number of clones) | (number of clones) |
| IL-1a (3) | GLP-1 (1) |
| IL-1b (3) | GLP-1-R (1) |
| IL-1-ra (3) | C1q (1) |
| IL-2 (3) | C1s (1) |
| IL-3 (3) | C3 (2) |
| IL-4 (4) | C4 (1) |
| IL-5 (3) | C5 (2) |
| IL-6 (4) | Factor B (1) |
| IL-7 (2) | B (1) |
| IL-8 (3) | Properdin (1) |
| IL-9 (3) | C1 esterase inhibitor (1) |
| IL-10 (3) | CD40 ligand (1) |
| IL-11 (3) | PSA (1) |
| IL-12 (4) | Leptin (1) |
| IL-13 (3) | LDL (2) |
| IL-16 (3) | Integrin α-10 (1) |
| IL-18 (3) | Integrin α-11 (1) |
| TGF-β1 (3) | Procathepsin (1) |
| TNF-α (3) | Tyrosine-protein kinase BTK (1) |
| TNF-β (4) | Tyrosine-protein kinase JAK3 (1) |
| INF-α (3) | B-lactamase (1) |
| VEGF (4) | Lewis^{x} (2) |
| Angiomotin (2) | Lewis^{y} (1) |
| MCP-1 (3) | B cell lymphoma AG (1) |
| MCP-3 (3) | Sialyl Lewis^{x} (1) |
| MCP-4 (3) | MUC-1 (1) |
| Eotaxin (3) | Streptavidin (1) (control) |
| RANTES (3) | Digoxin (1) (control) |
| GM-CSF (3) | FITC (1) (control) |
| CD40 (4) | TAT (2) (control) |

### Production and Processing of Antibody Microarrays

The production and handling of the antibody microarrays was performed according to a previous optimized set-up^{6,7,18,31}. Briefly, the scFv microarrays were fabricated, using a noncontact printer (Biochip Arrayer1, Perkin Elmer Life & Analytical Sciences, Wellesley, Mass., USA), which deposits approximately 330 pL/drop, using piezo technology. The scFv antibodies were arrayed by spotting 2 drops at each position and the first drop was allowed to dry out before the second drop was dispensed. The antibodies were spotted onto black polymer MaxiSorp microarray slides (NUNC A/S, Roskilde, Denmark), resulting in average 5 fmol scFv per spot (range 1.5-25 fmol). Eight replicates of each scFv-clone were arrayed to ensure adequate statistics. To assist the alignment of the grid during the subsequent quantification, a row containing Cy5 conjugated streptavidin (2 μg/ml) was spotted for every tenth row. In total, 160 antibodies and controls were printed per slide orientated in two columns with 8×80 spots per column. A hydrophobic pen (DakoCytomation Pen, DakoCytomation, Glostrup, Denmark) was used to draw a hydrophobic barrier around the arrays. The arrays were blocked with 500 μl 5% (w/v) fat-free milk powder (Semper AB, Sundbyberg, Sweden) in PBS overnight. All incubations were conducted in a humidity chamber at room temperature (RT). The arrays were then washed four times with 400 μl 0.05% Tween-20 in PBS (PBS-T), and incubated with 350 μl biotinylated serum diluted 1:10 (resulting in a total serum dilution of 1:450) in 1% (w/v) fat-free milk powder and 1% Tween in PBS (PBS-MT) for 1 h. Next, the arrays were washed four times with 400 μl PBS-T and incubated with 350 μl 1 μg/ml Alexa-647 conjugated streptavidin diluted in PBS-MT for 1 h. Finally, the arrays were washed four times with 400 μl PBS-T, dried immediately under a stream of nitrogen gas and scanned with a confocal microarray scanner (ScanArray Express, Perkin Elmer Life & Analytical Sciences) at 5 μm resolution using six different scanner settings. The ScanArray Express software V3.0 (Perkin Elmer Life & Analytical Sciences) was used to quantify the intensity of each spot using the fixed circle method. The local background was subtracted and to compensate for possible local defects, the two highest and two lowest replicates were automatically excluded, and each data point represents the mean value of the remaining four replicates. For protein analytes displaying saturated signals, values from lower scanner settings were scaled and used instead.

### Microarray Data Normalization

Chip-to-chip normalization of the data set was performed, using a semi-global normalization approach, conceptually similar to the normalization method used for DNA microarrays. The coefficient of variation (CV) was first calculated for each analyte over all samples and ranked. The 15% of the analytes displaying the lowest CV-values over all samples were identified, corresponding to 21 analytes, and used to calculate a chip-to-chip normalization factor. The normalization factor Ni was calculated by the formula Ni═Si/μ, where Si is the sum of the signal intensities of the 21 analytes for each sample, and μ is the average of Si from all samples. Each data set generated from one sample was divided with the normalization factor Ni. Log2 values were calculated for the signal intensities for all analytes and ranked using a Wilcoxon test.

### Microarray Data Analysis

The support vector machine (SVM) is a supervised learning method that we used to classify the samples as either healthy or breast cancer (FIG. 2). The supervised classification was performed using a linear kernel, and the cost of constraints was set to 1, which is the default value in the R function SVM, and no attempt was performed to tune it. This absence of parameter tuning was chosen to avoid overfilling. The SVM was trained using a leave-one-out cross validation procedure. Briefly, the training sets (n=40) were generated in an iterative process in which the samples were excluded one by one. The SVM was then asked to blindly classify the left out sample as either healthy or breast cancer, and to assign a SVM decision value, which is the signed distance to the hyperplane. No filtration on the data was done before training the SVM. Further, a receiver operating characteristics (ROC) curve, as constructed using the SVM

decision values and the area under the curve, was found. In those cases, where smaller cohorts of samples were compared (FIG. 3), significantly up- or down-regulated analytes (p<0.05) were identified using Wilcoxon test, log transformed and mean centred. The samples were then hierarchically clustered and visualized as a heat map, using Cluster and TreeView³³.

### Results

Evaluation of scFv Microarrays

We analyzed directly labelled, non-fractionated serum samples from metastatic breast cancer patients (n=20) and healthy controls (n=20), using a large-scale recombinant antibody microarray. The array was composed of 129 human recombinant scFv antibodies directed against 60 serum proteins, mainly of immunoregulatory nature (Table 3). A representative microarray image of a breast cancer serum is shown in FIG. 1A, demonstrating that dynamic signal intensities, homogenous and distinct spot morphologies, as well as high signal-to-noise ratios were obtained. The reproducibility of the set-up was validated by determining, the i) intraassay reproducibility, i.e. the spot-to-spot variation, (FIG. 1B), and ii) the inter-assay correlation, i.e. the reproducibility of duplicate experiments (same sample, but analyzed on different arrays) (FIG. 1C). In agreement with previous results6, the reproducibility was found to be high, with an intra-assay reproducibility of 0.99 and an inter-assay reproducibility of 0.96. The specificity and sensitivity (pM range) of the microarray set-up for targeting complex proteomes has previously been validated^{6, 7, 18, 31, 32}

### Classification of Metastatic Breast Cancer

To evaluate the ability of the microarray set-up to classify metastatic breast cancer patients based on a simple blood test, we examined the serum protein expression profile generated by all 129 antibodies included on the chip (FIG. 2 and Table 3). Consequently, we ran a leave-one-out cross validation, with a Support Vector Machine (SVM), and collected the decision values for each sample. In FIG. 2A, a Receiver Operating Characteristics (ROC) curve was constructed, using the decision values produced by SVM. The results showed that the metastatic breast cancer patients vs. healthy controls could be discriminated, displaying an area under the curve of 0.92.

The decision value is the output of the predictor, and samples with a prediction value below a threshold are predicted to be breast cancer. The threshold parameterizes the trade-off between sensitivity and specificity and is often set to zero. The 20 metastatic breast cancer samples obtained decision values in the interval of −3.64 to 0.26, and the healthy controls in the interval from −0.51 to 2.11 (FIG. 2B). Thus, with a threshold value of zero, the sensitivity and specificity was 85% in our data set. Notably, in the training and testing of the SVM, no filtration of data was performed, i.e. data from all analytes measured was included in the analysis.

Furthermore, the 11 highest ranked, i.e. significantly differentially expressed, analytes corresponding to 9 non-redundant serum analytes, are shown in FIG. 2B, suggesting a breast cancer-associated serum biomarker signature. When including only these highest ranking analytes in the analysis, the ROC area under the curve was increased to 0.97. This predictor signature allowed us to classify the metastatic breast cancer patients vs. healthy controls displaying a sensitivity and specificity of 95%, respectively (data not shown).

The tentative signature of the 11 top differentially expressed serum analytes contained both analytes previously associated with breast cancer, e.g. sialyl Lewis^{x 21, 23}, C3²², C4³⁴ and IL-8²⁴⁻²⁷, as well markers previously not observed in the disease, e.g. IL-5 and IL-7. The signal intensities observed for the top 3 differentially expressed analytes, including C4, IL-8 and C5, are shown in FIG. 2C. The results showed that the signal intensities in serum from breast cancer patients had increased 1.6 times (IL-8 and C5) and 3.6 times (C4). Of note, the observed differences in signal intensities can be interpreted in terms of relative changes of the amount of each individual analyte present. However, they do not necessarily reflect the magnitude of absolute changes for one analyte compared to another, due to the inherent limitations associated with direct labelling of different analytes in complex proteomes³⁵.

Effect of Anti-Inflammatory Drugs and/or Hormones

Screening serum samples of metastatic breast cancer patients, as well as other forms of cancers¹², will reflect not only directly cancer-related related affects, but also indirect systemic responses due to e.g. inflammatory-associated events. In addition, intake of various drugs may also affect the protein expression signatures observed. In an attempt to address this issue, we examined the expression profiles of only those metastatic breast cancer patients (n=6) and healthy controls (n=6) that had not taken any anti-inflammatory drugs and/or hormones at the time when the serum samples were collected. The differentially expressed serum analytes (p<0.05) in these two restricted cohorts of samples were identified, using a Wilcoxon test, and visualized as a heat map (FIG. 3A) based on unsupervised hierarchical clustering. The results showed that 8 serum proteins were identified that completely distinguished between metastatic breast cancer patients and healthy controls. Notably, only 2 of the analytes, C4 and IL-7, overlapped with the first signature, composed of 9 non-redundant analytes, which was generated by analyzing all samples.

### Sub-Classification of Metastatic Breast Cancer Patients

In an attempt to further stratify the metastatic breast cancer patients, we compared the known clinical parameters (Table 2), including ER status, PgR status, clinical stage, tumour volume, and histological features, with the observed serum protein expression profiles. No correlation could, however, be observed with respect to PgR status or clinical stages. Similarly, no correlation could be observed with respect to ER status, tumour volume, and histological features, although these analyses were hampered by the small sets of sample groups available. Of note, 5 serum proteins distinguishing between the most advanced clinical stages of the metastatic breast cancer patients, stages III and IV (n=8) vs. healthy controls (n=6), excluding those controls that had taken any inflammatory drugs and/or hormones, could be observed (FIG. 3B). Except for procathepsin, 4 of these 5 analytes overlapped with the observed signature distinguishing between non-treated (anti-inflammatory drugs and/or hormones) breast cancer patients and healthy controls.

### Additional Biomarkers Useful for Diagnosis of Breast Cancer

Further experiments also demonstrated differential expression levels (P<0.05) of TNF-b and IL-3 when comparing cohorts of metastatic breast cancer patients versus non-metastatic breast cancer patients (see FIG. 4).

### Discussion

Novel cancer biomarker signatures for early and improved detection and diagnostics, that in the long run also could be used to predict tumour relapses, monitor treatment, and stratify patients based on non-invasive set-ups are critical, since more than 11 million people are diagnosed with cancer every year¹⁻⁵. In this study, we have shown that large-scale recombinant scFv antibody

microarrays could provide an unique, miniaturized mean to perform classification of metastatic breast cancer, by multiplexed serum protein profiling of a blood sample. The results showed that the cancer patients could be classified with high sensitivity and specificity.

In comparison, antibody-based microarrays have previously been used to profile e.g. bladder cancer¹³, colon cancer¹⁷, lung cancer³⁶, liver cancer³⁷, ovarian cancer³⁸, pancreatic cancer¹², prostate cancer^{15,16} and squamous cell carcinoma¹⁴ (for review see refs 5, 10, 39). Albeit successful, outlining the potential of the technology within oncoproteomics^{5,10,39}, the ability of the biomarker signatures to distinguish between different carcinomas or between cancer and inflammation has been difficult to achieve, except in a few cases^{13,16,18,40}. To a great extent, this reflects the performance of the array set-ups, e.g. functionality, sensitivity, and range of antibody specificities^{5,10,39}, as well as the fact that the serum signature most likely will mirror both directly cancer-associated affects as well as indirect systemic affects. In the present study, the former issue has been minimized by adopting a proven recombinant scFv antibody microarray technology platform^{6,7}, enabling us to target high- as well as low-abundant immunoregulatory proteins in non-fractionated serum proteomes, thus providing several key advantages³¹. We found that the metastatic breast cancer patients could be classified with a specificity and sensitivity of 85% and a ROC=0.92, using all 129 analytes, corresponding to 60 non-redundant serum analytes. To make this tentative signature more manageable, we also chose to present a condensed signature, composed of the 11 most significantly differentially expressed analytes, which corresponded to 9 non-redundant biomarkers. Although additional screening will be required to validate this signature, our data showed that it was capable of classifying breast cancer with 95% specificity and sensitivity and a ROC=0.97. Of note, this 11 marker signature had only 1 of 14 (IL-5) analytes in common with one signature identifying Helicobacter pylori infected stomach tissue 18, and 4 of 35 (C3, C5, IL5-, and IL-7) in common with systemic lupus erythematosus, an autoimmune disorder with a significant inflammatory component (Wingren et al, manuscript in preparation). This indicated that the breast cancer signature was not related to general inflammation. In addition, the breast cancer signature was also different from that observed for e.g. bladder cancer¹³, lung cancer⁴⁰, pancreatic cancer¹² (Ingvarsson et al, submitted), and prostate cancer¹⁶. In the case of gastric adenoma carcinoma¹⁸, 7 of 28 (not C4 and II-5) biomarkers overlapped, indicating a similarity to this much larger signature, although it should be noted that tissue extracts and not serum samples were analyzed in that particular study.

The strength of the 11 analyte breast cancer signature was further highlighted by the fact that the breast cancer patients could be adequately classified, although they were individually treated with a wide range of therapeutic agents that might influence their serum signatures differently. In this context, it was of interest to note that an additional biomarker signature with a higher predictive power was indicated, when only those patients that had not taken anti-inflammatory drugs and/or hormones, were profiled. Although, larger sample cohorts need to be analyzed to validate these results, this second signature overlapped less with the gastricadenoma carcinoma signature¹⁸.

Among the 9 non-redundant analyte signature, 5 was up-regulated (sialyl Lewis^{x}, C3, C4, C5 and IL-8) and 4 down-regulated (TM peptide, IL-5, IL-7 and MCP-3). In agreement, increased levels of sialyl Lewis^{x}, a molecule of importance for the interaction between tumour cells and endothelium, have previously been observed for breast cancer patients^{21, 23}. Similarly, increased levels of truncated forms of C3a, originating from C3, have also been confirmed²². C3 is a versatile complement protein, supporting the activation of all three pathways of complement activation, and that has been suggested to function in immune surveillance against tumours, although the mechanisms for the latter remain unknown^{22, 41}. The role of the complement system was further highlighted by the increased serum levels of both C4 and C5, supported by early work of Lamoureux and co-workers ³⁴. In addition, the up-regulation of IL-8, suggested to be involved in breast cancer invasion and progression, have also been observed in several studies^{24-28,42}. Furthermore, IL8 is expressed by both breast carcinoma and stroma cells and has been implicated in tumour angiogenesis⁴³. The novel findings that serum levels of IL-5, IL-7 and MCP-3 were down-regulated could, for example, reflect a lowered tumour immune surveillance by eosinophils (IL-5)⁴⁴, impaired maintenance of T cell memory (IL-7)⁴⁵, and a reduced attraction of leukocyte subsets which potentially recognize and destroy tumour cells (MCP-3)⁴⁶. In contrast, IL-7 was shown to mediate tumour growth in vitro⁴⁷ and levels of IL7 expression in tumour tissue also correlated with tumour aggressiveness in breast cancer patients⁴⁸.

The panels of 8 biomarkers observed when profiling a focused cohort of breast cancer, where patients taking any anti-inflammatory drugs and/hormones had been excluded, showed an up-regulation of typical TH1 cytokines, e.g. TNF-β and IL-12, accompanied by a downregulation of TH2 cytokines, e.g. IL-4, indicating a TH1 skewing of the immune system. Increased levels of IL-12 and an induced TH1 response have previously also been observed in breast cancer tissue^{24,42,49}.

Additional experiments comparing cohorts of metastatic breast cancer patients vs non-metastatic breast cancer patients observed that TNF-β is downregulated in cancer patients and IL3- is upregulated in cancer patients.

In previous work, a relationship between a single clinical parameter, such as ER status, and the expression levels of e.g. IL-8, has been observed²⁴⁻²⁷. With the possible exception for the combined cohort of the most advanced forms of breast cancer, stages III and IV, we did not detect any such relationship, although these particular analyses were impaired by too small cohorts.

Taken together, in this first study, we have been able to classify metastatic breast cancer with a high specificity and sensitivity, based on a blood-test, using the novel approach of recombinant antibody microarray analysis.

### REFERENCES

1. Hu, S.; Loo, J. A.; Wong, D. T., Human body fluid proteome analysis. Proteomics 2006, 6, (23), 6326-53.
2. Levenson, V. V., Biomarkers for early detection of breast cancer: what, when, and where? Biochim Biophys Acta 2007, 1770, (6), 847-56.
3. Cho, W. C., Contribution of oncoproteomics to cancer biomarker discovery. Mol Cancer 2007, 6, 25.
4. Hanash, S., Disease proteomics. Nature 2003, 422, (6928), 226-32.
5. Borrebaeck, C. A., Antibody microarray-based oncoproteomics. Expert Opin Biol Ther 2006, 6, (8), 833-8.
6. Ingvarsson, J.; Larsson, A.; Sjöholm, A. G.; Truedsson, L.; Jansson, B.; Borrebaeck, C. A. K.; Wingren, C., Design of recombinant antiody microarrays for serum protein profiling: targeting of complement proteins. J Proteome Res 2007, 6, (9), 3527-3536.
7. Wingren, C.; Ingvarsson, J.; Dexlin, L.; Szul, D.; Borrebaeck, C. A. K., Design of recombinant antibody microarrays for complex proteome analysis: choice of sample labeling-tag and solid support. Proteomics 2007, 7, (17), 3055-3065.
8. Kingsmore, S. F., Multiplexed protein measurement: technologies and applications of protein and antibody arrays. Nat Rev Drug Discov 2006, 5, (4), 310-20.
9. Wingren, C.; Borrebaeck, C. A. K., Antibody microarrays: current status and key technological advances. Omics 2006, 10, (3), 411-27.
10. Wingren, C.; Borrebaeck, C. A. K., High-throughput proteomic using antibody microarrays—un update. Expert Review of Molecular Diagnostics 2007, In press.
11. Pavlickova, P.; Schneider, E. M.; Hug, H., Advances in recombinant antibody microarrays. Clin Chim Acta 2004, 343, (1-2), 17-35.
12. Orchekowski, R.; Hamelinck, D.; Li, L.; Gliwa, E.; vanBrocklin, M.; Marrero, J. A.; Vande Woude, G. F.; Feng, Z.; Brand, R.; Haab, B. B., Antibody microarray profiling reveals individual and combined serum proteins associated with pancreatic cancer. Cancer Res 2005, 65, (23), 11193-202.
13. Sanchez-Carbayo, M.; Socci, N. D.; Lozano, J. J.; Haab, B. B.; Cordon-Cardo, C., Profiling bladder cancer using targeted antibody arrays. Am J Pathol 2006, 168, (1), 93-103.
14. Knezevic, V.; Leethanakul, C.; Bichsel, V. E.; Worth, J. M.; Prabhu, V. V.; Gutkind, J. S.; Liotta, L. A.; Munson, P. J.; Petricoin Iii, E. F.; Krizman, D. B., Proteomic profiling of the cancer microenvironment by antibody arrays. Proteomics 2001, 1, (10), 1271-1278.
15. Miller, J. C.; Zhou, H.; Kwekel, J.; Cavallo, R.; Burke, J.; Butler, E. B.; Teh, B. S.; Haab, B. B., Antibody microarray profiling of human prostate cancer sera: antibody screening and identification of potential biomarkers. Proteomics 2003, 3, (1), 56-63.
16. Shafer, M. W.; Mangold, L.; Partin, A. W.; Haab, B. B., Antibody array profiling reveals serum TSP-1 as a marker to distinguish benign from malignant prostatic disease. Prostate 2007, 67, (3), 255-67.
17. Sreekumar, A.; Nyati, M. K.; Varambally, S.; Barrette, T. R.; Ghosh, D.; Lawrence, T. S.; Chinnaiyan, A. M., Profiling of cancer cells using protein microarrays: discovery of novel radiation-regulated proteins. Cancer Res 2001, 61, (20), 7585-93.
18. Ellmark, P.; Ingvarsson, J.; Carlsson, A.; Lundin, B. S.; Wingren, C.; Borrebaeck, C. A. K., Identification of protein expression signatures associated with Helicobacter pylori infection and gastric adenocarcinoma using recombinant antibody microarrays. Mol Cell Proteomics 2006, 5, (9), 1638-46.
19. Jain, K. K., Personalised medicine for cancer: from drug development into clinical practice. Expert Opin Pharmacother 2005, 6, (9), 1463-76.
20. Louhimo, J.; Finne, P.; Alfthan, H.; Stenman, U. H.; Haglund, C., Combination of HCGbeta, CA 19-9 and CEA with logistic regression improves accuracy in gastrointestinal malignancies. Anticancer Res 2002, 22, (3), 1759-64.
21. Kurebayashi, J.; Nomura, T.; Hirono, M.; Okubo, S.; Udagawa, K.; Shiiki, S.; Ikeda, M.; Nakashima, K.; Tanaka, K.; Sonoo, H., Combined measurement of serum sialyl Lewis X with serum CA15-3 in breast cancer patients. Jpn J Clin Oncol 2006, 36, (3), 150-3.
22. Li, J.; Orlandi, R.; White, C. N.; Rosenzweig, J.; Zhao, J.; Seregni, E.; Morelli, D.; Yu, Y.; Meng, X. Y.; Zhang, Z.; Davidson, N. E.; Fung, E. T.; Chan, D. W., Independent validation of candidate breast cancer serum biomarkers identified by mass spectrometry. Clin Chem 2005, 51, (12), 2229-35.
23. Matsuura, N.; Narita, T.; Mitsuoka, C.; Kimura, N.; Kannagi, R.; Imai, T.; Funahashi, H.; Takagi, H., Increased level of circulating adhesion molecules in the sera of breast cancer patients with distant metastases. Jpn J Clin Oncol 1997, 27, (3), 135-9.
24. Nicolini, A.; Carpi, A.; Rossi, G., Cytokines in breast cancer. Cytokine Growth Factor Rev 2006, 17, (5), 325-37.
25. Lin, Y.; Huang, R.; Chen, L.; Li, S.; Shi, Q.; Jordan, C.; Huang, R. P., Identification of interleukin-8 as estrogen receptor-regulated factor involved in breast cancer invasion and angiogenesis by protein arrays. Int J Cancer 2004, 109, (4), 507-15.
26. Lin, Y.; Huang, R.; Chen, L. P.; Lisoukov, H.; Lu, Z. H.; Li, S.; Wang, C. C.; Huang, R. P., Profiling of cytokine expression by biotin-labeled-based protein arrays. Proteomics 2003, 3, (9), 1750-7.
27. Lin, Y.; Wang, S. M.; Lu, W. M.; Huang, R. P., [Effect of interleukin-8 in cell invasion and proliferation of human breast cancer]. Zhonghua Wai Ke Za Zhi 2005, 43, (23), 1541-4.
28. Vazquez-Martin, A.; Colomer, R.; Menendez, J. A., Protein array technology to detect HER2 (erbB-2)-induced ‘cytokine signature’ in breast cancer. Eur J Cancer 2007, 43, (7), 1117-1124.
29. Smith, L.; Watson, M. B.; O'Kane, S. L.; Drew, P. J.; Lind, M. J.; Cawkwell, L., The analysis of doxorubicin resistance in human breast cancer cells using antibody microarrays. Mol Cancer Ther 2006, 5, (8), 2115-20.
30. Hudelist, G.; Pacher-Zavisin, M.; Singer, C. F.; Holper, T.; Kubista, E.; Schreiber, M.; Manavi, M.; Bilban, M.; Czerwenka, K., Use of high-throughput protein array for profiling of differentially expressed proteins in normal and malignant breast tissue. Breast Cancer Res Treat 2004, 86, (3), 281-91.
31. Wingren, C.; Borrebaeck, C. A. K., Antibody microarray-based proteomics. Bioforum Europe 2007, 5, 34-5.
32. Soderlind, E.; Strandberg, L.; Jirholt, P.; Kobayashi, N.; Alexeiva, V.; Aberg, A. M.; Nilsson, A.; Jansson, B.; Ohlin, M.; Wingren, C.; Danielsson, L.; Carlsson, R.; Borrebaeck, C. A. K., Recombining germline-derived CDR sequences for creating diverse single-framework antibody libraries. Nat Biotechnol 2000, 18, (8), 852-6.
33. Eisen, M. B.; Spellman, P. T.; Brown, P. O.; Botstein, D., Cluster analysis and display of genome-wide expression patterns. Proc Natl Acad Sci U S A 1998, 95, (25), 14863-8.
34. Lamoureux, G.; Mandeville, R.; Poisson, R.; Legault-Poisson, S.; Jolicoeur, R., Biologic markers and breast cancer: a multiparametric study-1. Increased serum protein levels. Cancer 1982, 49, (3), 502-12.
35. Ingvarsson, J.; Lindstedt, M.; Borrebaeck, C. A. K.; Wingren, C., One-step fractionation of complex proteomes enables detection of low abundant analytes using antibody-based microarrays. J Proteome Res 2006, 5, (1), 170-6.
36. Bartling, B.; Hofmann, H. S.; Boettger, T.; Hansen, G.; Burdach, S.; Silber, R. E.; Simm, A., Comparative application of antibody and gene array for expression profiling in human squamous cell lung carcinoma. Lung Cancer 2005, 49, (2), 145-54.
37. Zhou, H.; Bouwman, K.; Schotanus, M.; Verweij, C.; Marrero, J. A.; Dillon, D.; Costa, J.; Lizardi, P.; Haab, B. B., Two-color, rolling-circle amplification on antibody microarrays for sensitive, multiplexed serum-protein measurements. Genome Biol 2004, 5, (4), R28.
38. Mor, G.; Visintin, I.; Lai, Y.; Zhao, H.; Schwartz, P.; Rutherford, T.; Yue, L.; Bray-Ward, P.; Ward, D. C., Serum protein markers for early detection of ovarian cancer. Proc Natl Acad Sci U S A 2005, 102, (21), 7677-82.
39. Wingren, C.; Borrebaeck, C. A. K., High-throughput proteomics using antibody microarrays. Expert Rev Proteomics 2004, 1, (3), 355-64.
40. Gao, W. M.; Kuick, R.; Orchekowski, R. P.; Misek, D. E.; Qiu, J.; Greenberg, A. K.; Rom, W. N.; Brenner, D. E.; Omenn, G. S.; Haab, B. B.; Hanash, S. M., Distinctive serum protein profiles involving abundant proteins in lung cancer patients based upon antibody microarray analysis. BMC Cancer 2005, 5, 110.
41. Sahu, A.; Lambris, J. D., Structure and biology of complement protein C3, a connecting link between innate and acquired immunity. Immunol Rev 2001, 180, 35-48.
42. Chavey, C.; Bibeau, F.; Gourgou-Bourgade, S.; Burlinchon, S.; Boissiere, F.; Laune, D.; Rogues, S.; Lazennec, G., Oestrogen receptor negative breast cancers exhibit high cytokine content. Breast Cancer Res 2007, 9, (1), R15.
43. Ben-Baruch, A., Host microenvironment in breast cancer development: inflammatory cells, cytokines and chemokines in breast cancer progression: reciprocal tumor-microenvironment interactions. Breast Cancer Res. 2003 5, (1), 31-36.
44. Simson, L.; Ellyard, J. I.; Dent, L. A.; Matthaei, K. I.; Rothenberg, M. E.; Foster, P. S.; Smyth, M. J.; Parish, C. R., Regulation of carcinogenesis by IL-5 and CCL11: a potential role for eosinophils in tumor immune surveillance. J Immunol 2007, 178, (7), 4222-9.
45. Vudattu, N. K.; Magalhaes, I.; Schmidt, M.; Seyfert-Margolis, V.; Maeurer, M. J., Reduced numbers of IL-7 receptor (CD127) expressing immune cells and IL-7-signaling defects in peripheral blood from patients with breast cancer. Int J Cancer 2007.
46. Menten, P.; Wuyts, A.; Van Damme, J., Monocyte chemotactic protein-3. Eur Cytokine Netw 2001, 12, (4), 554-60.
47. Al-Rawi, M. A.; Rmali, K.; Mansel, R. E.; Jiang, W. G., Interleukin 7 induces the growth of breast cancer cells through a wortmannin-sensitive pathway. Br J Surg 2004, 91, (1), 61-8.
48. Al-Rawi, M. A.; Rmali, K.; Watkins, G.; Mansel, R. E.; Jiang, W. G., Aberrant expression of, interleukin-7 (IL-7) and its signalling complex in human breast cancer. Eur J Cancer 2004, 40, (4), 494-502.
49. Vitolo, D.; Ciocci, L.; Ferrauti, P.; Tiboni, F.; Cicerone, E.; Gallo, A.; De Vincentiis, M.; Baroni, C. D., Interleukin-12-related cytokine gene expression in carcinomas of the breast, lung, and larynx: a study at tissue level. Cancer Detect Prev 2000, 24, (5), 422-34.

1. A method for determining the presence of breast cancer cells in an individual comprising the steps of:
a) providing a serum or plasma sample to be tested;
b) determining a protein signature of the test sample by measuring the presence and/or amount in the test sample of two or more proteins selected from the group defined in Table 1a or 1b, which two or more proteins include IL-5 and/or MCP-3;
wherein the presence and/or amount in the test sample of the two or more proteins selected from the group defined in Table 1a or 1b is indicative of the presence of breast cancer cells.

2. The method according to claim 1 further comprising the steps of:
c) providing a control serum or plasma sample from a healthy individual;
d) determining a protein signature of the control sample by measuring the presence and/or amount in the control sample of the two or more proteins measured in step (b);
wherein the presence of breast cancer cells is identified in the event that the presence and/or amount in the test sample of the two or more proteins measured in step (b) is different from the presence and/or amount in the control sample.

3-10. (canceled)

11. The method according to claim 1 wherein step (b) comprises measuring the presence and/or amount in the test sample of all nine of the proteins defined in Table 1a or 1b.

12. The method according to claim 1 wherein an increase in one or more of C3, C4, C5, IL-8, IL-3 and sialyl Lewis^{x}, and/or a decrease in one or more of IL-5, IL-7, MCP-3, TNF-β and TM peptide is indicative of the presence of breast cancer cells in the individual.

13-15. (canceled)

16. The method according to claim 1 wherein the patient has not taken any anti-inflammatory drugs and/or hormones prior to providing the serum or plasma sample.

17. The method according to claim 1 wherein step (b) comprises determining a protein signature of the test sample by measuring the presence and/or amount in the test sample of one or more proteins selected from the group consisting of TNF-β, IL-12, C4, MCP-1, IL-3, IL-7, integrin α10 and IL-4.

18. (canceled)

19. The method according to claim 17 wherein step (b) comprises determining a protein signature of the test sample by measuring the presence and/or amount in the test sample of all of the proteins from the group consisting of TNF-β, IL-12, C4, MCP-1, IL-3, IL-7, integrin α10 and IL-4.

20. (canceled)

21. The method according to claim 2 wherein step (b) and/or step (d) is performed using a first binding agent capable of binding to the two or more proteins.

22. The method according to claim 21 wherein the first binding agent comprises or consists of an antibody or an antigen-binding fragment thereof.

23.-26. (canceled)

27. The method according to claim 2 wherein the two or more proteins in the test sample are labelled with a detectable moiety prior to performing step (b) and/or step (d).

28. (canceled)

29. (canceled)

30. The method according to claim 2 wherein step (b) and/or step (d) are performed using an array.

31. (canceled)

32. (canceled)

33. The method according to claim 30 wherein the array is selected from the group consisting of microarray; microarray; and nanoarray.

34. The method according to claim 2 wherein step (b) and/or step (d) are performed using an assay comprising a second binding agent capable of binding to the one or more proteins or detectable moiety thereon, the second binding agent also comprising a detectable moiety.

35-38. (canceled)

39. The method according to claim 34 wherein the second binding agent comprises or consists of an antibody or an antigen-binding fragment thereof or wherein the second binding agent comprises or consists of streptavidin.

40-42. (canceled)

43. An array for determining the presence of breast cancer cells, the array comprising two or more first binding agents as defined in claim 21.

44. The array according to claim 43 wherein the first binding agents are capable of binding to all of the proteins defined in Table 1a or 1b.

45-47. (canceled)

48. A diagnostic kit for determining the presence of breast cancer cells comprising:
A) an array according to claim 43; and, optionally,
B) instructions for performing methods of determining the presence of breast cancer cells.

49. The diagnostic kit according to claim 48 further comprising one or more second binding agents capable of binding to the one or more proteins or detectable moiety thereon, the second binding agent also comprising a detectable moiety.

50. The diagnostic kit according to claim 48 further comprising one or more controls samples.

51. (canceled)

52. (canceled)


SEQUENCE LISTING (2 sequences)

SEQ ID NO: 1 - protein, 242 aa, Homo sapiens
MAEVQLLESGGGLVQPGGSLRLSCAASGFTFSSYGFHWVRQAPGKGLEWVSLISWDGGSTYYADSVKGRFTISRDNSKNTLYLQMNSLRAEDTAVYYCARGTWFDPWGQGTLVTVSSGGGGSGGGGSGGGGSQSVLTQPPSASGTPGQRVTISCSGSSSNIGNNAVNWYQQLPGTAPKLLIYRNNQRPSGVPDRFSGSKSGTSASLAISGLRSEDEADYYCAAWDDSLSWVFGGGTKLTVLG

SEQ ID NO: 2 - protein, 31 aa, Artificial Sequence
  Affinity tag
DYKDHDGDYKDHDIDYKDDDDKAAAHHHHHH